(19) United States
(12) Patent Application Publication
(10) Pub. No.: US 2022/0257919 A1
(43) Pub. Date: Aug. 18, 2022

(54) VALVING SYSTEM WITH IMPROVED FLUSHABILITY AND METHODS OF USING SAME

(71) Applicant: NP Medical Inc., Clinton, MA (US)

(72) Inventor: John Damarati, Marlborough, MA (US)

(73) Assignee: NP Medical Inc., Clinton, MA (US)

(21) Appl. No.: 17/628,456

(22) PCT Filed: Aug. 2, 2019
(86) PCT No.: PCT/US2019/044825

§ 371 Date: Jan. 19, 2022

(51) Int. Cl. A61M 39/24; A61M 39/02

(52) U.S. Cl. CPC A61M 39/24; A61M 39/0247; A61M 2039/242; A61M 2039/0258; A61M 2039/0282; A61M 2039/027

(57) ABSTRACT

A valving device includes a body with an inlet, an outlet and an internal fluid path extending from the inlet to the outlet. Within the internal fluid path, the valving device has a valve mechanism with an open mode that allows fluid flow through the valve mechanism between the inlet and outlet and closed mode that prevents fluid flow through the valve mechanism. The valve mechanism has a first surface facing the inlet and a second surface facing the outlet. The body at least partially defines a flow corral. The flow corral(s) is/are located distal to the valve mechanism and redirect fluid passing through the valving device back toward the second surface of the valve mechanism to flush the underside of the valve mechanism.

### TECHNICAL FIELD

The present invention relates to valving systems, and more particularly to diaphragm based valving devices and systems with improved flushability.

### BACKGROUND ART

In instances in which a patient will need regular administration of fluid or medications (or regular withdrawal of fluids/blood), catheters are often inserted into the patient and used to administer the fluids/medications. The catheter may remain in the patient for extended periods of time (several hours to several days or longer). Additionally, an extension tube, an administration set, or both may be connected to the catheter to facilitate use of the catheter and connection of a medical implement (e.g., a syringe).

The extension tube, administration set, medical implement, or similar vascular access device may include a medical valving device. In general terms, medical valving devices often act as a port that may be repeatedly accessed to non-invasively inject fluid into (or withdraw fluid from) a patient's vasculature. Consequently, a medical valve permits the patient's vasculature to be freely accessed without requiring such patient's skin to be repeatedly pierced by a needle. The medical valve may be a luer activated valve (with or without a swabable septum) and/or a pressure activated valve (similarly with or without a swabable septum). An issue with many prior art medical valves is that, as fluid flows through the valve (e.g., from an inlet to the outlet of the valve), fluid may stagnate in various areas within the valve. For example, in diaphragm based pressure activated valves, fluid may stagnate on the underside of the diaphragm where the diaphragm is supported within the valve. This stagnated fluid is difficult to clear/flush out which, in turn, reduces the utility of these prior art medical valves.

### SUMMARY OF THE INVENTION

In accordance with one embodiment of the invention, a valving device may include a body defining the structure of the device, a valve mechanism and at least one flow corral. The body may have an inlet, an outlet and an internal fluid path extending from the inlet to the outlet. The valve mechanism may be located within the internal fluid path and may have an open mode that allows fluid flow through the valve mechanism between the inlet and outlet and closed mode that prevents fluid flow through the valve mechanism. The valve mechanism may have a first surface facing the inlet and a second surface facing the outlet. The flow corral(s) may be defined, at least in part, by a portion of the body and may be located distal to the valve mechanism. The flow corral may redirect fluid passing through the valving device from the inlet to the outlet toward the second surface of the valve mechanism. This, in turn, may flush the underside of the valve mechanism.

In some embodiments, the valve mechanism may be a pressure activated valve, and may transition from the closed mode to the open mode in the presence of a forward pressure directed from the inlet to the outlet. The body may have a seating surface, and the first surface of the valve mechanism may seal against the seating surface when in the closed mode. Additionally or alternatively, the valve mechanism may include an aperture extending through it. The aperture may open in the presence of a backward/retrograde pressure from the outlet toward the inlet.

The body may have a base portion that (1) extends radially inward from an inner wall of the body and (2) is distal to the valve mechanism. A plurality of support arms may extend proximally from the base portion. The support arms may support the valve mechanism within the internal fluid path and/or may bias the valve mechanism towards the closed mode. The support arms may be spaced from one another to form flow channels between each of the support arms. The flow channels may allow fluid flow between each of the plurality of support arms. The valve mechanism may deform over the support arms as the valve mechanism transitions from the closed mode to the open mode.

In accordance with further embodiments, each of the support arms may have an angled radially outward face that, at least in part, forms the flow corral(s) and redirects the fluid passing through the valving device toward the second surface of the valve mechanism. Additionally or alternatively, the body may include angled radially inward faces located within the inner wall of the body. The angled radially inward faces may also, at least in part, form the flow corral(s). The angled radially inward faces may be recessed into the inner wall of the body and may be located proximal to the base portion.

The angled radially outward surfaces may be oriented at a first angle and the angled radially inward faces may be oriented at a second angle. The first angle may oppose the second angle. The first and second angles may be acute angles relative to a longitudinal axis of the body and/or obtuse relative to the base portion. The angled radially inward faces may be aligned with one of the angled radially outward faces. The body may include an inlet body and an outlet body. The inlet may be located in the inlet body and the outlet may be located in the outlet body. The inlet may connect to a tube of an extension set and/or vascular access device.

In accordance with additional embodiments, a vascular access device (e.g., an extension set) may include a valving device as described above, a tube and a female luer connector. The tube may have a first end and a longitudinal portion and may be fluidly connected to the inlet of the valving device at the first end. The female luer may be connected to the longitudinal portion, and the tube may fluidly connect the female luer connector and the inlet of the valving device.

In accordance with further embodiments, a vascular access device may include a valving device as described above, a tube, a medical device and/or male luer, and a female luer connector. The tube may have a first end and a second end. The medical device and/or male luer connector may be located at the first end of the tube and may be configured to connect to the inlet of the valving device. The female luer connector may be connected to the second end of the tube. The tube may fluidly connect the female luer connector and the inlet of the valving device

In accordance with additional embodiments, a method for transferring fluid through a valving device may include providing a valving device as described above and fluidly connecting a medical implement to the inlet of the body. The method may then apply a forward pressure on the valving device. The forward pressure may transition the valve mechanism from the closed mode to the open mode. The method may then transfer fluid through the valving device using the medical implement. The fluid may flow into the inlet, through the internal fluid path, around the valve mechanism and out the outlet. The flow corral may redirect at least a portion of the fluid back toward the second surface of the valve mechanism to flush the underside.

### BRIEF DESCRIPTION OF THE DRAWINGS

The foregoing features of embodiments will be more readily understood by reference to the following detailed description, taken with reference to the accompanying drawings, in which:

FIG. 1 schematically shows a perspective view of a pressure activated valve in accordance with various embodiments of the present invention;

FIG. 2A schematically shows a cross-section view of the valve shown in FIG. 1 in the closed mode, in accordance with some embodiments of the present invention;

FIG. 2B schematically shows a cross-section view of the valve shown in FIG. 1 in the open mode, in accordance with some embodiments of the present invention;

FIGS. 3A-3C schematically show various perspective exploded views of the pressure activated valve shown in FIG. 1, in accordance with some embodiments of the present invention;

FIG. 4 schematically shows a top view of a bottom portion of the valve shown in FIG. 1, in accordance with various embodiments of the present invention;

FIG. 5 schematically shows a bottom view of a top portion of the valve shown in FIG. 1, in accordance with various embodiments of the present invention;

FIGS. 6A-6C schematically show the valve mechanism within the valve of FIG. 1 during operation, in accordance with some embodiments of the present invention;

FIG. 7A schematically shows an extension set with a pressure activated valve, in accordance with embodiments of the present invention;

FIG. 7B schematically shows a cross-sectional view of the pressure activated valve on the extension set, in accordance with embodiments of the present invention; and

FIG. 8 schematically shows a cross-sectional view of an alternative pressure activated valve in accordance with further embodiments of the present invention.

### DETAILED DESCRIPTION OF SPECIFIC EMBODIMENTS

In illustrative embodiments, a valving device (e.g., a medical valve) has an internal valve mechanism located within an internal fluid path of the valving device. The housing of the valve has one or more “flow corrals” that redirect the fluid flowing through the valve back toward the valve mechanism after it has passed the valve mechanism. This, in turn, helps with valve flushing. Details of illustrative embodiments are discussed below.

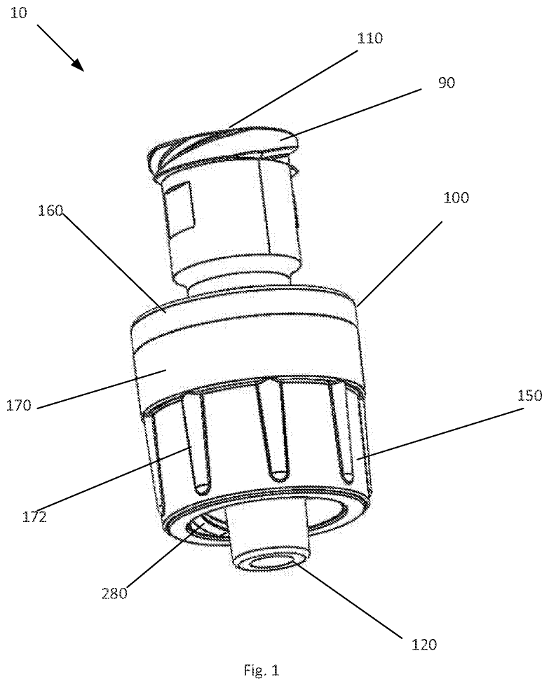

FIG. 1 schematically shows a perspective view of a medical valve 10 in accordance with some embodiments of the present invention. The valve 10 has a housing 100 forming an interior having a proximal port 110 (e.g., an inlet) for receiving a medical instrument (not shown), and a distal port 120 (e.g., an outlet). The valve 10 has an open mode that permits fluid flow through the valve 10, and a closed mode that prevents fluid flow through the valve 10. To that end, the interior contains a valve mechanism that selectively controls (i.e., allow/permits) fluid flow through the valve 10. The fluid passes through a complete fluid path that extends between the proximal port 110 and the distal port 120.

It should be noted that although much of the discussion herein refers to the proximal port 110 as an inlet, and the distal port 120 as an outlet, as discussed in greater detail below and in some embodiments, the proximal and distal ports 110 and 120 also may be respectively used as outlet and inlet ports. Discussion of these ports in either configuration therefore is for illustrative purposes only.

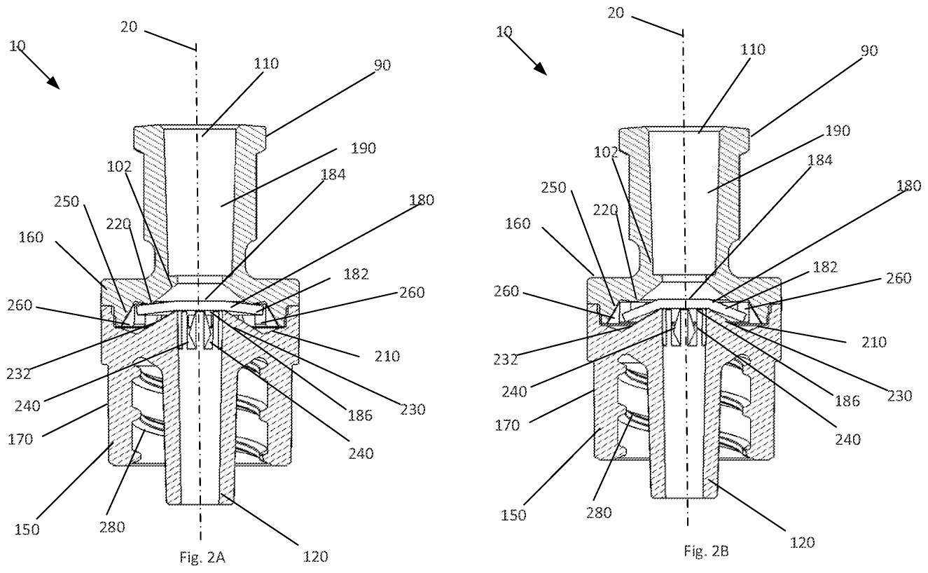

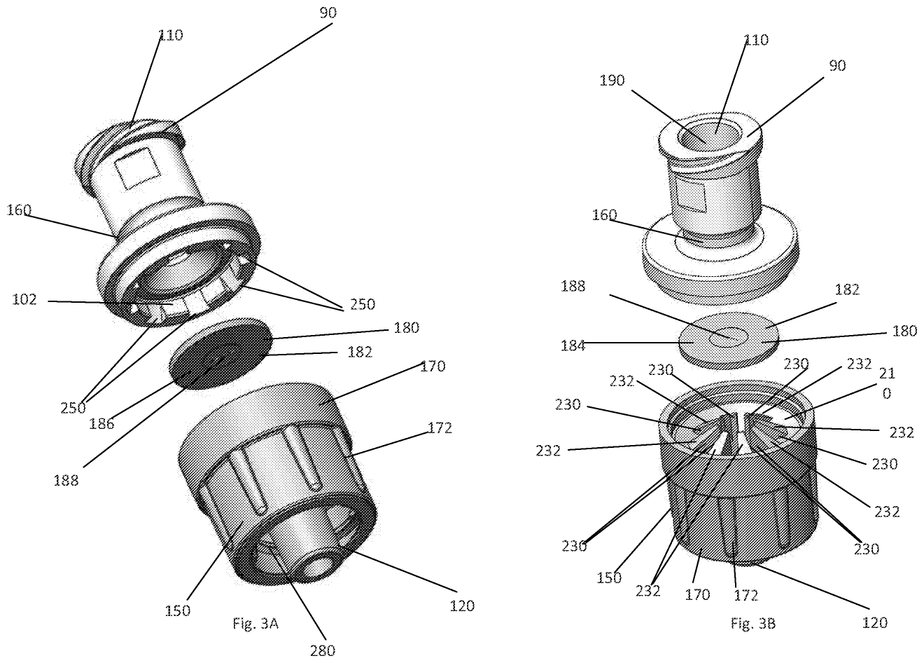

The outside surface of the valve proximal port 110 may have inlet threads 90 for connecting a medical instrument (not shown). Alternatively or in addition, the proximal end may have a slip design for accepting instruments that do not have a threaded interconnect. In a similar manner, the distal end of the valve 10 has a skirt 150 containing threads 280 (see FIG. 3) for connecting a threaded port of a catheter or a different medical instrument, to the valve distal port 120. The skirt 150 may also include ribs 172 that allow a medical practitioner to easily grasp and handle the valve 10. The proximal end inlet threads 90 and the distal end threads 280 preferably comply with ANSI/ISO standards (e.g., they are able to receive/connect to medical instruments complying with ANSI/ISO standards). In addition to the threads described above, the internal geometry of the inlet housing 160 (e.g., shown in FIGS. 2A-2B and 3A-3C, discussed below) may have a taper and comply with ANSI/ISO standards.

FIG. 2A schematically shows the cross section of the valve shown in FIG. 1 when in the closed mode and FIG. 2B schematically shows the cross section of the valve in the open mode. As shown, the housing 100 includes an inlet housing 160 and an outlet housing 170, which connect together to form the interior of the medical valve 10. Within the interior, the medical valve 10 has a valve mechanism 180 located within the fluid path 190 through the housing 100. The inlet housing 160 and the outlet housing 170 may be joined together in a variety of ways, including a snap-fit connection, ultrasonic welding, plastic welding, or other method conventionally used in the art.

Within the interior, the body/housing 100 may have an inner wall 102 that extends along at least a portion of the longitudinal axis 20 of the valve 10. The inner wall 102 forms/defines the internal fluid path 190 that extends through the valve 10 from the inlet 110 to the outlet 120. As discussed in greater detail below, the body/housing 100 (e.g., the outlet body/housing 170) may have a base portion 210 that extends inward from the inner wall 102. It should be noted that, although the fluid path 190 is shown as having a circular cross-sectional shape, other embodiments may have fluid paths with different cross-sectional shapes.

As noted above, to control fluid flow through the valve 10, the interior of the body/housing 100 may include a valve mechanism 180 within the internal fluid path 190. For example, the valve 10 may include a pressure activated valve 180 (PAV) that includes a diaphragm 182 (e.g., a flat diaphragm; FIGS. 3A-3C). Alternatively, at least a portion of the diaphragm 182 may have a curvature. When in the closed mode (FIG. 2A), the valve mechanism 180 (e.g., the top surface 184 of the diaphragm 182) may seal against a seating surface 220 within the interior of the body/housing 100 (e.g., on the inner wall 102 of the body/housing 100). The valve mechanism 180 prevents fluid flow through the body/housing 100 (e.g., through the internal fluid path 190) until it is exposed to a large enough pressure (e.g., a forward pressure directed from the inlet 110 toward the outlet 120) to deform the diaphragm 182 (FIG. 2B) and allow fluid to pass through the valve 10.

In some embodiments, the valve mechanism 180 may be a two-way pressure activated valve. In such embodiments, the diaphragm 180 may include an aperture/slit 188 that extends through the diaphragm 180 (e.g., from the top surface/side 184 to the bottom surface/side 186). In a manner similar to the functionality of the diaphragm 180, in the presence of a sufficient backward/retrograde pressure (e.g., a cracking pressure directed from the outlet 120 toward the inlet 110), the aperture/slit 188 may open to allow fluid to flow from the outlet 120 toward the inlet 110 through the internal fluid path 190 and aperture/slit 188. It is important to note that a diaphragm 180 and slit 188 configuration should be chosen such that the patient's venous pressure is below the retrograde/backward (i.e. proximally-directed) cracking pressure of the valve mechanism 180 to prevent the venous pressure from opening the slit 188/pressure activated valve 180. Although a diaphragm 180 with a slit 188 may achieve the functionality of a two-way pressure activated valve, other two-way PAVs known in the art may also be used within the body/housing 100. The forward pressure required to deform the diaphragm 180 and the cracking pressure of the aperture/slit 188 (e.g., the backward/retrograde pressure required to open the aperture/slit 188) may depend on the application. However, in some embodiments, the forward pressure required to deform the diaphragm 180 may be less than the cracking pressure of the aperture/slit 188.

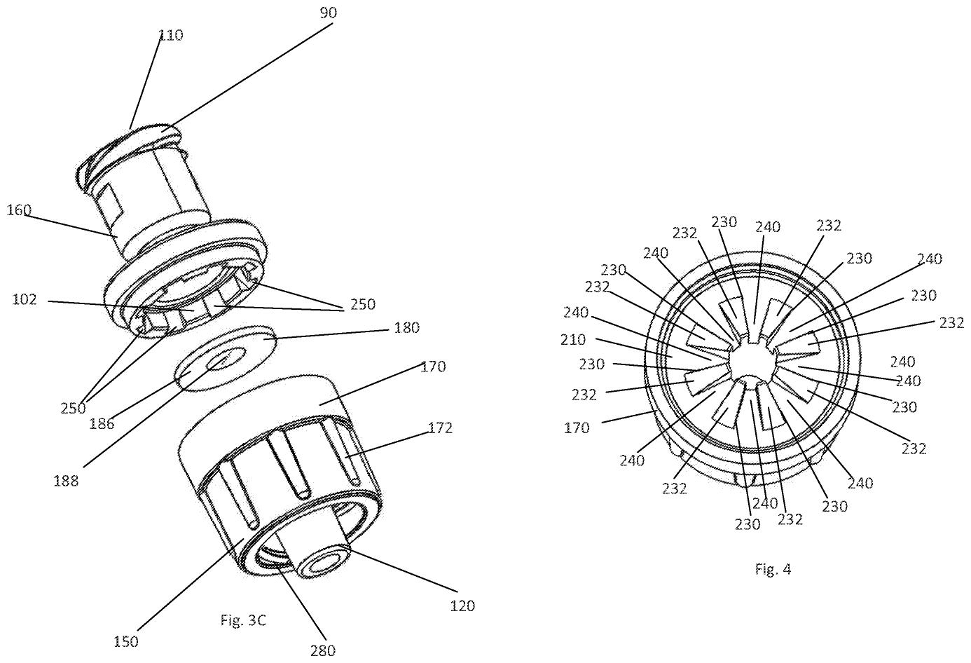

To help support the valve mechanism 180 within the fluid path 190, the valve 10 may include a number of support arms 230 (FIG. 4) that extend proximally from the base portion 210. The support arms 230 may normally contact the bottom surface 186 of the diaphragm 182 to support the valve 180 within the flow path 190 and bias the diaphragm 182 toward the closed mode such that the first/top surface 184 of the diaphragm 182 contacts/seals against the seating surface 220. Although any number of configurations and lengths may be used for the support arms 230, in some embodiments, the support arms 230 may each have a similar length and width and may have angled radially outward face 232. To allow fluid flow between the support arms 230, the support arms 230 may be spaced from one another to create channels 240 between them. It should be noted that although FIG. 3B shows eight support arms 230, other embodiments may utilize more or less than eight arms 230. In addition to the number of support arms 230, the width, length and spacing of the support arms 230 can vary based on the size of the internal fluid path 190 and the desired flow properties of the valve 10 (of system in which the valve 10 is incorporated).

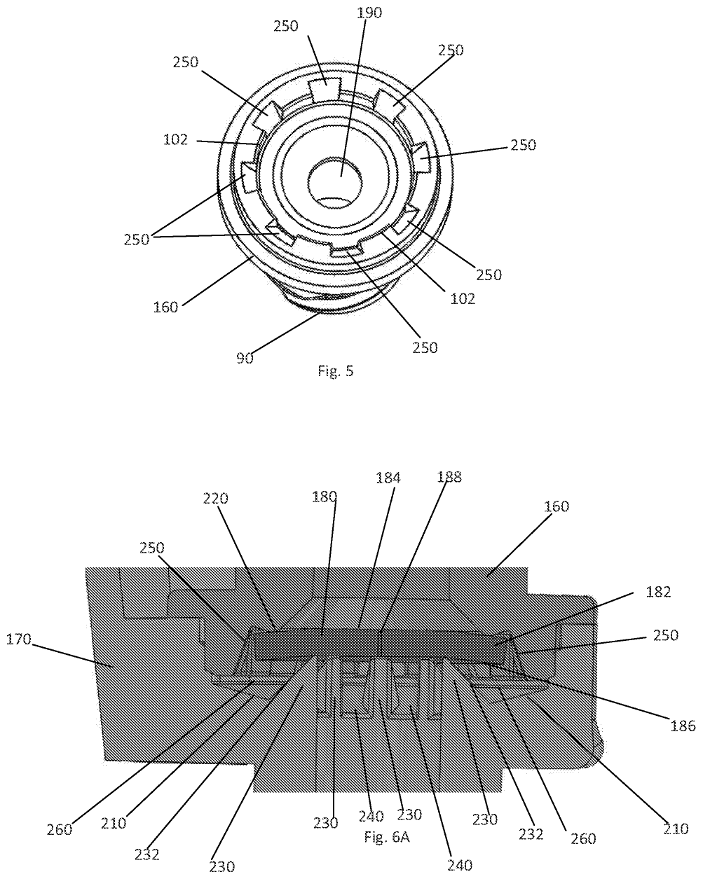

Located radially outward from the valve member 180 and proximal to the base portion 210, the housing/body 100 may also include a number of angled faces 250 (FIG. 5) that are formed within (e.g., recessed within) the wall 102 of the body/housing 100. These angled faces 250 may be inwardly facing (e.g., with respect to the longitudinal axis 20) and may be radially aligned to and have a length and/or width that generally corresponds with each of the angled radially outward faces 232. As noted above, the valve 10 may have flow corrals that help improve flushing within the valve 10. To that end, the angled faces 250 (e.g., the angled radially inward faces), a portion of the base 210, and the angled radially outward faces 232 on the support arms 230 form a number of flow corrals 260 that circulate the fluid flowing around the valve mechanism 180 back towards the second side/bottom surface 186 (i.e. underside) of the valve mechanism 180 such that the redirected fluid flushes the fluid path region at the corresponding support arm. For example, each of the angled faces 250, a corresponding angled radially outward faces 232 and an adjacent portion of the base 210 may form a flow corral 260. Therefore, the number of flow corrals 260 may depend on the number of support arms 230 and angled faces 250 within the valve 10. It should be noted that the wall 102 of the body 100 in between the angled faces 250 may produce a surface that aids with the centering of the valve mechanism 180 during assembly and operation of the valve 10.

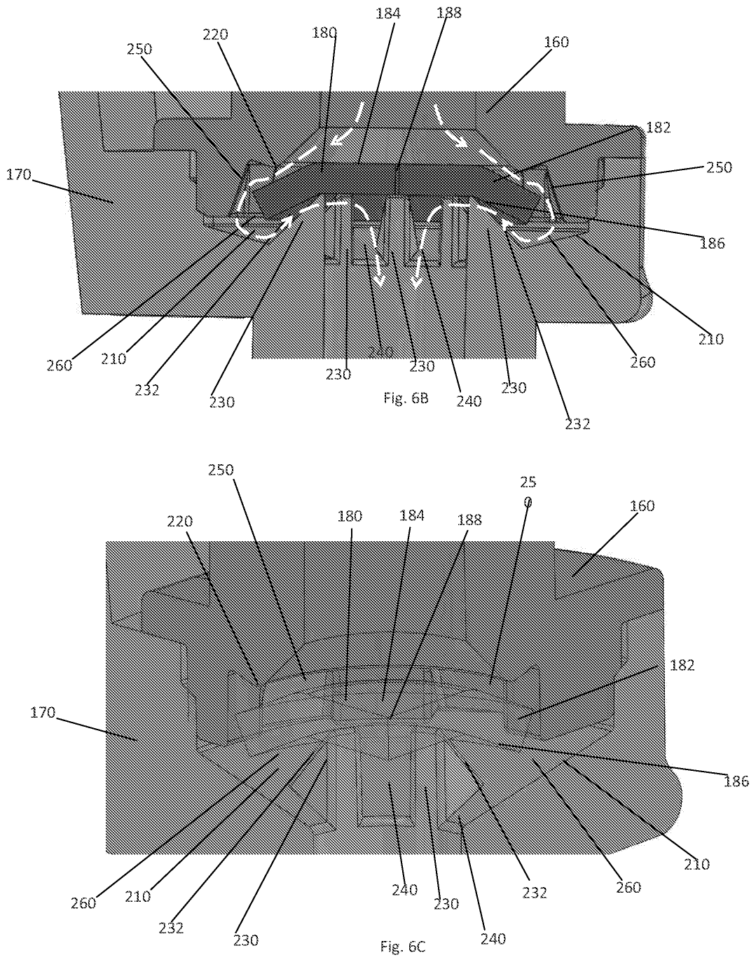

It is important to note that the angles of the angled radially inward faces 250 and the angled radially outward faces 232 may depend on the application and the amount of flushing required. For example, in some embodiments, the angled radially inward faces 250 and the angled radially outward faces 232 may have opposing acute angles relative to the longitudinal axis 20 of the body 100 of less than 60 degrees (e.g., between 35 and 45 degrees). Alternatively, the angled faces 250 and the angled radially outward faces 232 may also be oriented at different/multiple angles instead of a single angle relative to the longitudinal axis 20 of the body 100. For example, different angled faces 250 and angled radially outward faces 232 may be oriented at different angles and/or the angled faces 250 may be oriented at a different angle as compared to the radially outward faces 232. Additionally or alternatively, the angled faces 250 and the angled radially outward faces 232 may each form slightly obtuse angles with the base 210. In this manner, the flow corrals 260 may form a generally U-shaped flow circulation path (FIG. 6B) that, in turn, further organizes fluid flow towards the second side 186 of the valve mechanism 180. It should be noted that, although the angled faces 250 and the angled radially outward faces 232 are shown as having flat surfaces, arcuate and other contours (e.g., concave, convex, U-shaped, V-shaped, etc.) are envisioned within the scope of the present invention. Furthermore, the distal end of the angled faces 250 may be located at/near the foot of the angled radially outward faces in a manner that does not require base 210 to form flow corrals 260.

FIG. 6A shows a cross-sectional close up of the valve mechanism 180 when the valve 10 is in the closed mode. FIGS. 6B and 6C show cross-sectional close-ups of the valve mechanism 180 when in the open mode and the fluid flow past the valve mechanism 180 when transferring fluid to the patient. For example, during operation, the user may connect a medical implement (e.g., a needleless syringe) to the inlet 110 of the valve 10 and begin to inject fluid. The forward pressure created by this fluid will cause the valve mechanism 180 to deform and cause the periphery of the valve mechanism 180 to move away from the seating surface 220 on the wall 102 of the body 100 and bend about the support arms 230. This, in turn, allows the fluid to flow from the first end of the body 100 (e.g., the inlet 110), past the seating surface 220 and around the valve mechanism 180 into the flow corrals 260. Once the fluid reaches the flow corrals 260, the flow corrals 260 redirect the fluid back towards the second side/bottom surface 186 of the valve mechanism 180, through the space/channels 240 between each of the support arms 230, and then towards the second end/outlet 120 of the body 100. As discussed above, by redirecting the fluid back toward the second side/bottom 186 of the valve mechanism 180, the flow corrals (e.g., formed by the angled faces 250, angled radially outward faces 232, and optionally base 210) improve valve flushing, particularly, distal to the valve mechanism 180.

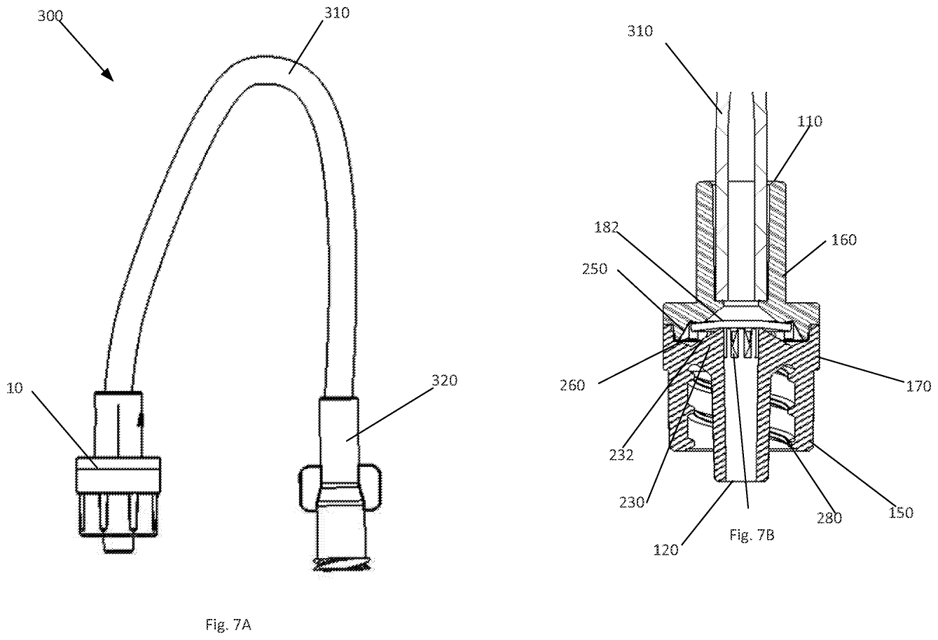

It should be noted that the valve 10 may be incorporated into any number of peripheral flow valving systems used within IV Therapy and Vascular Access devices. For example, as shown in FIGS. 7A and 7B, the valve 10 may be incorporated into an extension set 300. In such embodiments, the inlet 110 of the valve 10 may not have the inlet threads 90 or be tapered to receive a medical instrument. Rather, a tube 310 of the extension set 300 may be inserted into and secured within the inlet 110 or alternatively, inserted into and secured within another component (e.g., a medical device and/or male luer connector) that in turn is connected to the inlet 110. For example, the tube 310 may be press-fit, ultrasonic welded, plastic welded, etc. within the inlet 110 or the component. During use, the medical implement (e.g., a needleless syringe) may be connected to a female luer 320 located on a longitudinal end of the tube 310 and the fluid may be injected into the valve 10 via the tube 310 and female luer connector 320.

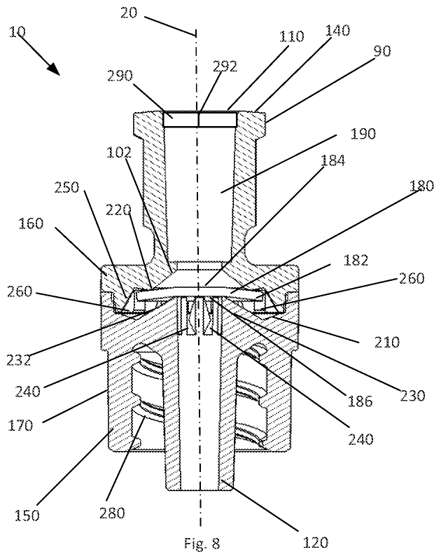

Although the embodiments described above show a valve having an open inlet 110, other embodiments may include proximal gland 290 that provides a low pressure seal within the inlet 110. The proximal gland 290 may have a resealable aperture 292 that extends entirely through its profile. The aperture 292 may, for example, be a pierced hole or a slit. Alternatively, the proximal gland 290 may be molded with the aperture 292. When the valve 10 is in the closed mode, as shown in FIG. 8, the aperture 292 may be held closed by the inner surface of the housing 100. In that case, the inner diameter of the housing 100 at the proximal/inlet port 110 may be smaller than the outer diameter of the proximal gland 290 and thus, the housing 100 squeezes the aperture 292 closed. Alternatively, the gland 290 may be formed so that the aperture 292 normally stays closed in the absence of radially inward force provided by the inner diameter of the proximal port 110. In other words, the proximal gland 290 is formed so that the aperture 292 normally is closed. The proximal gland 290 may be generally flush with or extend slightly above the exterior inlet face 140 of the inlet housing 160. The proximal gland 290 and the exterior inlet face 140 thus present a swabbable surface, i.e., it may be easily wiped clean with an alcohol swab, for example, or other swab. Such valves typically have been referred to in the art as “swabbable valves.”

It should be understood that by incorporating the flow corrals 260 discussed above, various embodiments of the present invention are able to improve the flushability of pressure activated valves. For example, the flow corrals 260 allow the user to fully clear a first fluid (e.g. blood) from the fluid path 190 with a second fluid (e.g. saline) using a minimal flush volume.

The embodiments of the invention described above are intended to be merely exemplary; numerous variations and modifications will be apparent to those skilled in the art. All such variations and modifications are intended to be within the scope of the present invention as defined in any appended claims.

1. A valving device comprising:
a body defining the structure of the valving device, the body having an inlet and an outlet and an internal fluid path extending from the inlet to the outlet, the body also having a base portion that extends radially inward from an inner wall of the body and a plurality of angled radially inward faces;
a valve mechanism located within the internal fluid path and proximal to the base portion, the valve mechanism having an open mode that allows fluid flow through the valve mechanism between the inlet and outlet and a closed mode that prevents fluid flow through the valve mechanism, the valve mechanism having a first surface facing the inlet and a second surface facing the outlet;
a plurality of support arms extending proximally from the base portion, the plurality of support arms supporting the valve mechanism within the internal fluid path, each of the plurality of support arms having an angled radially outward face, each of the plurality of angled radially inward faces generally aligned with one of the angled radially outward faces;
at least one flow corral defined, at least in part, by the plurality of angled radially inward faces and the angled radially outward faces, the at least one flow corral located distal to the valve mechanism, the flow corral configured to redirect at least a portion of the fluid passing through the valving device from the inlet to the outlet toward the second surface of the valve mechanism, thereby flushing an underside of the valve mechanism.

2. A valving device according to claim 1, wherein the valve mechanism is a pressure activated valve, the pressure activated valve configured to transition from the closed mode to the open mode in the presence of a forward pressure directed from the inlet to the outlet.

3. A valving device according to claim 1, wherein the body includes a seating surface, the first surface of the valve mechanism sealing against a seating surface when in the closed mode.

4. A valving device according to claim 1, wherein the valve mechanism includes an aperture extending therethrough, the aperture configured to open in the presence of a backward pressure from the outlet toward the inlet.

5. A valving device according to claim 4, wherein the backward pressure required to open the aperture is greater than a venous pressure.

6. A valving device according to claim 4, wherein the valve mechanism is configured to transition from the closed mode to the open mode in the presence of a forward pressure directed from the inlet to the outlet, the forward pressure required to transition the valve mechanism being less than the backward pressure required to open the aperture.

7. A valving device according to claim 1, wherein the plurality of support arms bias the valve mechanism towards the closed mode.

8. A valving device according to claim 1, wherein the plurality of support arms are spaced from one another, thereby forming flow channels between each of the support arms, the flow channels allowing fluid flow between each of the plurality of support arms.

9. A valving device according to claim 1, wherein the valve mechanism is configured to deform over the plurality of support arms as the valve mechanism transitions from the closed mode to the open mode.

10. A valving device according to claim 1, wherein the plurality of angled radially inward surfaces may be at least one selected from the group consisting of flat, angled, arcuate, concave, convex, U-shaped, and V-shaped.

11. A valving device according to claim 1, wherein the plurality of angled radially outward surfaces may be at least one selected from the group consisting of flat, angled, arcuate, concave, convex, U-shaped, and V-shaped.

12. A valving device according to claim 1, wherein the plurality of angled radially inward faces are recessed into the inner wall of the body.

13. A valving device according to claim 1, wherein the angled radially outward surfaces are oriented at a first angle and the angled radially inward faces are oriented at a second angle, the first angle opposing the second angle.

14. A valving device according to claim 13, wherein the first and second angles are acute angles relative to a longitudinal axis of the body.

15. A valving device according to claim 13, wherein at least one of the first and second angles is obtuse relative to the base portion.

16. A valving device according to claim 1, further comprising a septum located within the inlet, the septum having a septum aperture extending therethrough.

17. A valving device according to claim 1, wherein the body includes an inlet body and an outlet body, the inlet located in the inlet body and the outlet located in the outlet body.

18. A valving device according to claim 1, wherein the inlet is configured to connect to a tube of a vascular access device.

19. A vascular access device comprising:
a valving device having:
a body defining the structure of the valving device, the body having an inlet and an outlet and an internal fluid path extending from the inlet to the outlet, the body also having a base portion that extends radially inward from an inner wall of the body and a plurality of angled radially inward faces,
a valve mechanism located within the internal fluid path and proximal to the base portion, the valve mechanism having an open mode that allows fluid flow through the valve mechanism between the inlet and outlet and a closed mode that prevents fluid flow through the valve mechanism, the valve mechanism having a first surface facing the inlet and a second surface facing the outlet,
a plurality of support arms extending proximally from the base portion, the plurality of support arms supporting the valve mechanism within the internal fluid path, each of the plurality of support arms having an angled radially outward face, each of the plurality of angled radially inward faces generally aligned with one of the angled radially outward faces,
at least one flow corral defined, at least in part, by the plurality of angled radially inward faces and the angled radially outward faces, the at least one flow corral located distal to the valve mechanism, the flow corral configured to redirect at least a portion of the fluid passing through the valving device from the inlet to the outlet toward the second surface of the valve mechanism, thereby flushing an underside of the valve mechanism;
a tube having a first end and a longitudinal portion, the tube connected to the inlet of the valving device at the first end; and
a female luer connected to the longitudinal portion of the tube, the tube fluidly connecting the female luer connector and the inlet of the valving device.

20. A vascular access device comprising:
a valving device having:
a body defining the structure of the valving device, the body having an inlet and an outlet and an internal fluid path extending from the inlet to the outlet, the body also having a base portion that extends radially inward from an inner wall of the body and a plurality of angled radially inward faces,
a valve mechanism located within the internal fluid path and proximal to the base portion, the valve mechanism having an open mode that allows fluid flow through the valve mechanism between the inlet and outlet and a closed mode that prevents fluid flow through the valve mechanism, the valve mechanism having a first surface facing the inlet and a second surface facing the outlet,
a plurality of support arms extending proximally from the base portion, the plurality of support arms supporting the valve mechanism within the internal fluid path, each of the plurality of support arms having an angled radially outward face, each of the plurality of angled radially inward faces generally aligned with one of the angled radially outward faces,
at least one flow corral defined, at least in part, by the plurality of angled radially inward faces and the angled radially outward faces, the at least one flow corral located distal to the valve mechanism, the flow corral configured to redirect at least a portion of the fluid passing through the valving device from the inlet to the outlet toward the second surface of the valve mechanism, thereby flushing an underside of the valve mechanism;
a tube having a first end and a second end;
a medical device and/or male luer connector located at the first end of the tube and configured to fluidly connect to the inlet of the valving device; and
a female luer connected to the second end of the tube, the tube fluidly connecting the female luer connector and the medical device and/or male luer connector.

21. (canceled)
